(19) United States
(12) Patent Application Publication
(10) Pub. No.: US 2018/0047121 A1
(43) Pub. Date: Feb. 15, 2018

(54) Healthcare Delivery Computer Systems and Methods

(71) Applicant: Liveclinic Inc., Irvine, CA (US)

(72) Inventor: Joydeep Bhattacharyya, Irvine, CA (US)

(21) Appl. No.: 15/492,699

(22) Filed: Apr. 20, 2017

Related U.S. Application Data
(60) Provisional application No. 62/374,948, filed on Aug. 15, 2016.

(51) Int. Cl. G06Q 50/24; G06Q 10/02; G06Q 10/06; G06Q 50/22

(52) U.S. Cl. CPC G06Q 50/24; G06Q 50/22; G06Q 10/02; G06Q 10/06

(57) ABSTRACT

Systems and methods provide for a computer system that includes, one or more processors and non-transitory machine readable storage media coupled to the one or more processors and having instructions stored therein that are executable by the one or more processors. The processor configured to receive, by a location beacon, a signal from a mobile device that includes a device specific identifier and location specific identifier, determine an identity of an owner of the mobile device, update electronic health record with a location specific identifier of the mobile device and generate a notification message for the owner of mobile device and an alert for other individuals based on information regarding the owner of the mobile device.

### PRIORITY CLAIM RELATED APPLICATION

This application claims the benefit of and priority from U.S. Provisional Patent application No. 62/374,948 filed on Aug. 15, 2016, entitled as “Healthcare Delivery Computer Systems and Methods”, which is incorporated herein by reference in its entirety.

### BACKGROUND

The advent of mobile computing has increased efficiencies in various fields such as, finance, entertainment, e-mail, text messages and telephone phone calls. However, other fields such as clinics and other healthcare providers continue to use paper forms and other non-electronic methods for managing their practice. Improvements in clinical practice could be beneficial and lead to an effectual patient experience.

### SUMMARY

The following presents a simplified summary in order to provide a basic understanding of aspects of the systems and methods described herein. This summary is not intended to be complete summary of the various aspects of the systems and methods. The following summary describes the problems solved and possible solutions.

Aspects of this disclosure are directed to providing an integrative platform that tracks patient flow and workflow process in real time. The data from the processes is recorded automatically and transparently utilizing indoor navigation technology in conjunction with a web-based electronic healthcare record (EHR) application and cloud-based computing infrastructure. Recorded data is configured to be queried, manipulated, and analyzed directly through the EHR interface as a way of identifying operational inefficiencies, thereby presenting opportunities for increased cost-savings while streamlining patient throughput, and improving both patient and staff satisfaction.

Many navigation systems exist in order to help people find their way within a variety of environments. Global Positioning System (GPS) navigation, for instance, is a widely available method of navigating outdoors in areas where a clear view of the sky and satellite communication is available. GPS signals are often unsuitable for navigating indoors or enclosed structures such as healthcare clinics because the presence of walls and roofs interfere with, weaken GPS reliability is affected by factors such as signal availability and strength, and reflect GPS signals.

In recent years, many supplementary and alternative indoor navigation systems have been developed as a means of supplementing GPS capabilities. Examples of these include real-time locating systems (RTLS) that employ such technologies as radio frequency (RF) communication, optical infrared (IR) communication, acoustic (ultrasound) communication, and various other technologies, working either independently or collaboratively in various combinations. Common applications of these technologies include, but are not limited to, patient tracking, personnel tracking, room assignments, and asset tracking.

The significance of implementing RTLS within the clinical environment, therefore, cannot be understated. In order to accommodate ever-increasing patient volumes, healthcare facility administrators should increase their clinic's physical capacity by either expanding existing facilities and/or hiring more providers and support personnel, both of which involve considerable investments in terms of time and money. On the other hand, RTLS provides administrators with the means to increase patient throughput by identifying and presenting methods for improving workflow efficiency, ultimately allowing them to do more with less. Improvements in workflow efficiency through automation not only saves money, but increases patient safety by reducing administrative errors, and improves patient satisfaction by reducing the amount of time spent waiting during an appointment.

Most current RTLS systems require infrastructure that is costly to implement, require users to wear specialized tags, e.g. RFID tags, and measure only the relative position of the wearer in terms of their approximate distance from an RTLS, e.g., radio, receiver. In most cases, a patient is required to check in at the front desk of a clinic where they are issued a tag, bracelet, or some other form of transmitter, which is then worn by the patient, and subsequently returned at the conclusion of the appointment.

Furthermore, while some of these RTLS systems offer the option to integrate their RTLS management software with a facility's existing EHR, they are still themselves separate systems designed to solve a single problem, further fragmenting the healthcare system at a time when what is needed are more integrative solutions. The solution to the current issues is a real-time locating system that is physically un-intrusive to the user, relatively inexpensive to implement, that automates patient flow and clinical workflow processes, and which functions as a seamless extension to an EHR in such a way that healthcare administrators are able to make more informed decisions about how to manage clinical operations efficiently.

A method for establishing a bidirectional electronic communication between a payer (e.g. health insurance company) and a patient at the point of care where a patient is seen by a medical provider using an Electronic Health Record System. Triggering an electronic communication ability between payer and patient based on the real time assessment by a medical provider. The computer system and the electronic module working together enables the bidirectional communication.

Embodiments include system and method for managing healthcare delivery processes within a clinical environment using an integrated indoor navigation and web-based electronic health record platform.

### BRIEF DESCRIPTION OF THE DRAWINGS

These and other characteristics of the present invention will be more fully understood by reference to the following detailed description in conjunction with the drawings (attached), in which:

FIG. 1 is a schematic diagram illustrating the overall interconnectivity of the various components of the computer system;

FIG. 2A depicts the various functions performed by a patient through a patient device.

FIG. 2B is a set of process flow diagrams representing an example interaction of various components of the system from FIG. 1.

FIG. 2C is a set of process flow diagrams representing an example interaction of various components of the system from FIG. 1.

FIG. 2D is a set of process flow diagrams representing an example interaction of various components of the system from FIG. 1.

FIG. 2E is a set of process flow diagrams representing an example interaction of various components of the system from FIG. 1.

FIG. 2F is a set of process flow diagrams representing an example interaction of various components of the system from FIG. 1.

FIG. 2G is a process flow diagram representing an example interaction between various components of the system from FIG. 1.

FIG. 2H is a process flow diagram representing an example interaction between various components of the system from FIG. 1.

FIG. 2I is a process flow diagram representing an example interaction between various components of the system from FIG. 1.

FIG. 3A is a process flow diagram representing an example interaction between various components of the system from FIG. 1.

FIG. 3B is a process flow diagram representing an example interaction between various components of the system from FIG. 1.

FIG. 3C is a process flow diagram representing an example interaction between various components of the system from FIG. 1.

FIG. 4 is a schematic diagram representing an example healthcare facility from FIG. 1.

### DETAILED DESCRIPTION

As described, the system comprises several components arranged in a various configuration to maximize efficiency of communication between the components. The system itself is designed to be scalable according to the needs and operational capacity of the particular healthcare facility. The system includes the web-based electronic health record (EHR) application, which is accessible through a variety of computing devices such as desktop computers equipped with a web browser, as well through native mobile applications installed on mobile computing devices such as smartphones and tablets. Bluetooth Low Energy (BLE) location beacons are employed as a means of communicating location information to patient and provider devices equipped with BLE-compatible radio chips. These beacons are deployed in varying densities in strategic locations within a healthcare facility. A cloud-based infrastructure comprising a server and EHR database configured to receive information from patient and provider devices via the Internet by way of cellular networks, wired and wireless routers, and local area networks (LANs) of varying size and configuration. It is also possible, though not required, to integrate into the system wireless, i.e. BLE-compatible, medical devices such as digital blood pressure monitors, thermometers, height and weight scales, heart rate monitors, etc.

As described, the various implementations interact using various digital communication techniques. The wireless location beacons and wireless medical devices (if utilized) communicate with properly equipped mobile devices by way of Bluetooth Low Energy (BLE) radio transmitters. Mobile devices such as smartphones, notepads, and tablets, communicate with the cloud-based infrastructure by way of cellular signals and/or wireless local area networks (WLANs). The cloud-based infrastructure, likewise, sends and receives information to and from patient and provider devices via the internet by way of cellular networks, wired and wireless routers, local area networks (LANs) and WANs of varying size and configuration.

As described herein, how well the various elements comprising the system communicate with other elements depends entirely on such factors as available battery power, consistent and quality access to the internet via cellular networks, LANs, and WANs, and the reliability of cloud-infrastructure components and other mobile and stationary computing devices, and the dependability of web-based and native mobile applications. For instance, BLE location beacons, though conservative in terms of power consumption, are commonly powered using “button” batteries which must be tested and changed periodically. Mobile devices such as smartphones and tablets, particularly those belonging to patients, require steady access to cellular data networks. If for whatever reason a particular mobile device does not function properly or is otherwise disconnected from the cellular network, the device will be unable to communicate with the cloud-based infrastructure. Mobile device operability also largely depends on available battery power, as these devices must be recharged from time to time. Stationary computing devices, such as desktop computers and laptops, depend on steady access to LANs and WLANs as a means of accessing and the Internet and communicating with the cloud-based computing infrastructure. System crashes are also possible, potentially affecting communication between the server, database, and web-based and mobile applications, as all software applications are prone to malfunction.

As described, the components of the present invention interact with one another using primarily wireless technology. Examples of such technology utilized by the present invention include:

Bluetooth Low Energy (BLE) wireless location beacons and medical devices. Utilize short-wavelength UHF radio waves in the ISM band from 2.4 to 2.485 GHz. Range is power dependent and customizable, but typical max range is around 100 meters.

Wi-Fi transmitters/receivers installed in mobile devices, other wireless-capable computing devices, and wireless routers. Mainly utilize the 2.4 gigahertz (12 cm) UHF and 5 gigahertz (6 cm) SHF ISM radio bands. Range is typically around 100 meters, but depends on the frequency band, radio power output, antenna gain and antenna type as well as the modulation technique. Line-of-sight is the thumbnail guide but reflection and refraction can have a significant impact, especially indoors.

Mobile Internet connection utilizing a cellular service provider subscription. Utilize frequencies in the UHF radio frequency band allocated to mobile usage. Range is power dependent and depends largely on the availability of the subscriber network, device proximity to network nodes, e.g. cell towers, and the particular make and model of the device.

As described, the present disclosure comprises multiple components, each of which are available commercially and interact utilizing conventional means of communication. For instance, electronic health records (EHRs) have existed in one form or another over the past several decades as a means of electronically storing patient health information in a digital format.

The significance of implementing RTLS within the clinical environment, therefore, cannot be understated. In order to accommodate ever-increasing patient volumes, healthcare facility administrators are left with little choice but to increase their clinic's physical capacity by either expanding existing facilities and/or hiring more providers and support personnel, both of which involve considerable investments in terms of time and money. On the other hand, RTLS provides administrators with the means to increase patient throughput by identifying and presenting methods for improving workflow efficiency, ultimately allowing them to do more with less. Improvements in workflow efficiency through automation not only saves money, but increases patient safety by reducing administrative errors, and improves patient satisfaction by reducing the amount of time spent waiting during an appointment.

A cheaper and more versatile method of indoor navigation has recently emerged taking advantage of 2.4 GHz Bluetooth Low Energy (BLE) beacon technology. BLE transmissions require relatively little power, and BLE beacons can survive on a typical watch battery for over a year. Additionally, most all modern smartphones come equipped with an accelerometer, which is a tiny sensor that is able to detect changes in velocity as well as spatial orientation. Considering the near ubiquity of smartphones, and how nearly all new models of smartphone come equipped with BLE-compatible radio chips and accelerometers, it makes sense that these technologies have already been leveraged to create more accurate, and, therefore, more versatile, indoor navigation systems.

As described, the present invention differs from present technology, not in terms of the individual components comprising it, but in terms of the way these various components interact as an integrated whole as means of providing a more satisfying and streamlined customer experience, all the while automating many traditionally manual administrative processes and collecting and storing valuable information which can then be queried and analyzed for the purpose of identifying operational inefficiencies.

Healthcare delivery management solutions may include the various embodiments of RTLS technology and similar indoor navigation systems, as well as a wide array of EHR and practice management solutions, including both local client/server arrangements as well as cloud-based server solutions. However, currently available solutions are fragmented, leaving customers to invest in separate RTLS and EHR systems, with even further investment required should the customer seek to integrate these systems. The present invention maintains an advantage over current solutions because it integrates an interoperable web-based EHR, BLE beacon-based indoor navigation/tracking system, practice management functionality, analytics and reporting features, and workflow analysis and automation capabilities within a single, cloud-based server platform that is relatively inexpensive to adopt and easy to maintain.

As described, the present disclosure is designed specifically for use within a healthcare delivery environment, more specifically in a free-standing clinical outpatient (ambulatory) setting, but may also be configured and scaled to function within a variety of larger, more patient-dense environments such as hospitals or primary care centers.

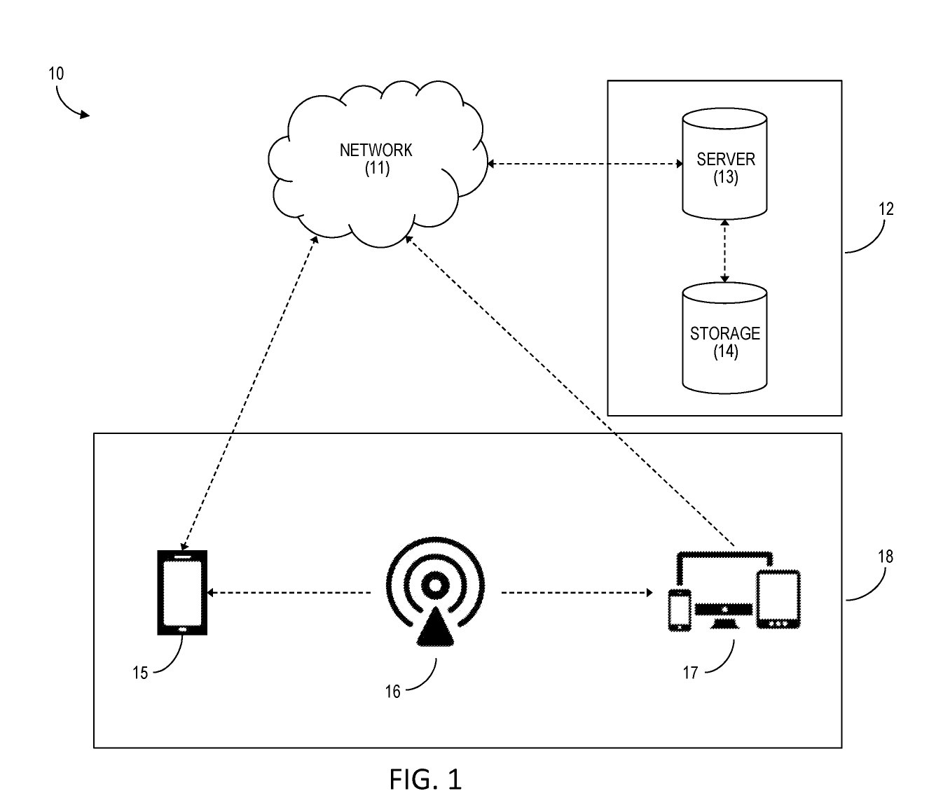

FIG. 1 depicts a system 10 for interacting with and integrating patients and patient data into the software (e.g., clinical) workflow. Specifically, FIG. 1 depicts a system for identifying, checking in, tracking, and assigning priority to patients of a healthcare facility 18, e.g. outpatient clinic, and thereby providing a more personalized patient experience, without requiring that they proactively or responsively provide personal information, such as a name or other identifying information, to an administrative assistant directly upon arrival at the facility 18. Furthermore, the patient is not required to complete any paperwork upon arrival at the facility 18, but will be automatically prompted instead to update their personal electronic health record (EHR) information privately and securely by submitting data to a secure server 13 via an application stored on their mobile computing device 15, e.g. smartphone, tablet, etc. Additionally, FIG. 1 depicts a system 10 for identifying and tracking the location and availability of healthcare facility personnel (e.g., doctors, physician assistants, nurses, medical assistants, etc.), as well as the availability of certain facility resources, e.g. exam rooms, etc., so as to efficiently optimize patient flow and clinical work flow. Depending on the nature of the patient's appointment (e.g., annual physical exam, injury, reported illness, etc.), requisite personnel are assigned accordingly as they become available or based on when the personnel are predicted to be available. The system 10 includes a cloud-based computing infrastructure 12 having at least one server 13 and at least one storage device 14 in inter-operable communication, forming the cloud-based computing infrastructure 12. The cloud-based computing infrastructure 12 is in communication with, and accessible through a network 11. In some embodiments, the cloud-based computing infrastructure 12 may communication with one or more health insurance company or their affiliate's server. In some embodiments, the identity of the health insurance company may be provided by the patient device 203 and sent to health insurance company server. The health insurance company server may be configured to receive diagnosis related information from the patient device 203, provider support device 206, provider device 207 and the office devices 205. Upon receiving the diagnosis related information, the health insurance company or their affiliate server may provide to the patient device 203 and the office device 205 information regarding treatment options that are covered and the cost of the patient. Health Insurance Company or their affiliates may contact the patient directly based on the diagnosis information for managing patient disease state In other embodiments, as the decisions are made on Diagnosis, the health insurance company or their affiliate's server may communicate directly with the officer device 205, provider support device 206, or provider device 207 regarding alternate treatment options that are free or of less expensive to the patient. However, all decisions regarding treatments option are always made by the healthcare provider. In various embodiments, the information provided by the health insurance company or their affiliate may assist managing the patient disease.

The network 11 can be, for example, an interconnected computer and/or telecommunications network that uses standard protocols, such as TCP/IP, or the like, to link a plurality of computing devices via a broad array of electronic, wireless, and optical networking technologies, known to those of skill in the art. For example, the network can be the Internet. The plurality of devices linked with the network can include a patient device 15. The patient device 15 can be a mobile computing device such as a smartphone, or any such similar mobile device (e.g., tablet, laptop, etc.) employing Bluetooth Low Energy (BLE)-equipped wireless technology. In addition, the system can include a healthcare facility 18 including one or more computing devices 17, and one or more location beacons 16. The facility computing devices 17 can be computing devices as discussed with respect to the computing infrastructure 12 and/or the patient device 15 and can be operable to communicate over the network 11. The facility computing devices 17 can be a combination of standalone desktop, laptop, or mobile computing devices each configured to communicate over the network 11.

Location beacons 16 are deployed in strategic locations within the healthcare facility 18 and operate in association with the facility 18. The healthcare facility 18 can be a brick and mortar or other such similar healthcare service provider, where outpatient healthcare is the primary service provided, and range in size as determined by patient load (e.g., doctor's offices, clinics, urgent care centers, ambulatory surgery centers, hospitals, etc.). The location beacons 16 can make use of wireless technology (i.e., Bluetooth Low Energy) and, as already mentioned, are likely positioned in various specific locations in and around the facility 18 (e.g., waiting room, exam rooms, physician offices, radiology labs, etc.). Location beacons 16 periodically transmit data wirelessly including a preprogrammed and universally unique identifier specific to that location within the facility 18 within a nearby range as would be understood by those of skill in the art familiar with BLE devices and ranges. For example, every 3 seconds the location beacon 16 can wireles sly transmit data packets containing the unique identifier for that facility and specific location within that facility. The wirelessly transmitted data can be received by any BLE-compatible computing devices 15, and 17, which can support an application, an operating system, or the like, e.g. the patient smartphone or similar mobile device. In accordance with an example embodiment, the patient device 15 is operating an application that is compatible with such a location beacon 16. In the present embodiment, the process described makes use of an application to interact with the data. It should be noted that the present disclosure is not intended to be limited only to the example embodiments making use of applications (whether native, proprietary, or otherwise), and that the present disclosure is intended to include any such tool operating on a patient device 15 that is BLE-compatible and has the ability to interact with the incoming data to effect the functionalities and features described herein, as would be appreciated by those of skill in the art. Accordingly, a native proprietary application can respond to, e.g., a data packet originating from an indoor location beacon 16 using the iBeacon protocol by Apple, Inc., the Eddystone protocol by Google, Inc., or other similar device, and can make use of the data in the manner described herein using the example of the application.

When the application operating on the patient device 15 receives/discovers the data from the location beacon identifying the healthcare facility 18, the application operating on the patient device 15 transmits identifying information about the patient (e.g. the device's International Mobile Station Equipment Identity (IMEI)) and the identification of the facility 18 to the server 13. The information regarding the patient can be stored locally on the patient device 15 associated with the application, or it can be stored remotely at other data storage 14 locations accessible by the patient device 15 or the cloud-based server 13. In response to receiving the information about the patient and the identification of the facility, the server 13 queries the patient's profile stored in the EHR database 14 and transmits the patient and facility information data to the facility computing device 17, via the network 11 (or at a minimum transmits the patient information to the facility computing device 17, using the facility information to identify where to transmit the patient information). The transmission of patient and facility information data to the facility computing device 17 can occur through either a push notification or alert, or other means known in the art, as would be appreciated by those of skill in the art. The above processes can be performed instantly and transparently from the perspective of the user. The patient and facility information data provides to the facility computing device 17 any information the patient operating the patient device 15 wishes to convey. For example, the patient and facility information data can include the patient's name, address, contact information, photograph, and other personal identifying information, physician preferences, patient allergy information or other medical condition information, information relating to historical visits by the patient device 15 (and therefore the patient) to the facility 18, and any other such information desired to be conveyed.

It should be noted that the patient device 15 is the device executing an application on behalf of the patient. In some embodiments, the application can be an application specifically tailored to carry out the functions of the present invention or the application can be a non-specific application configured to receive and/or utilize the information received from the location beacon(s) 16. The application executing on the patient device 15 can be executed on multiple different devices owned or operated by the patient, historically and into the future, where the relevant patient data can be attached to a login and account accessible by the application, such that the patient can access the application and the relevant data on numerous computing devices.

It should further be noted that as utilized herein, processes that involved transmitting information and data from one device to another can be implemented utilizing a number of technologies. For example, transmitting information to the patient device 15, the server 13, to the facility associated computing devices 17, as referred to in various steps discussed herein, can be implemented in accordance with the present disclosure utilizing such technologies as those for messaging, texting, chat, email, instant messaging, notifications, alerts, and the like, including any such technologies useful for communication between two or more devices over a wireless network 11, including the internet, cellular networks, or otherwise, as would be appreciated by those of skill in the art and readily implemented in accordance with the teachings of the present application.

When the patient and healthcare facility information data is received by the facility computing device 17, information is displayed on the facility computing device 17 to at least show the presence of the patient and the patient device 15 in the facility (i.e., a display shows when a patient, whether a walk-in or by appointment, by name, has arrived and wishes to be seen by a physician), and may initially show some basic patient information, such as the patient's name and/or appointment details. Based upon receiving patient information, the server 13 can automatically determine the availability of healthcare personnel (e.g., physicians, nurse practitioners, medical assistants, etc.) and facility resources (e.g., exam rooms, physicians' offices, etc.) then prioritize and allocate these personnel and facility resources as needed by the patient. When a patient arrives for their prearranged appointment, the server 13, which can actively track the physical location and status (i.e., those personnel available or unavailable to see a patient) of relevant healthcare personnel, can assign those personnel to the patient as they become available or as they are about to become available based on the current progress in their current task(s). As each exam room, physician's office, and waiting room within the healthcare facility is fitted with a uniquely identified location beacon 16, the cloud-based server 13 can be periodically or constantly updated with real-time information regarding the physical whereabouts of patients and healthcare personnel. The server 13 may use the real-time information to determine the vacancy, occupancy, and availability of the various rooms. Furthermore, the information generated and collected from each of these various components, being organized and stored within a database 14, can be analyzed statistically in the form of metrics, and viewed graphically by the user in the form of predefined reports and digital dashboard representations. Such reports and representations can be accessed by select facility personnel to determine where inefficiencies in facility work flow and patient flow exist, providing the information needed to identify and reduce, or altogether eliminate, such inefficiencies.

Applications operating on the facility associated computing devices 17 further enable additional data or information to be entered relating to the patient as associated with the patient device 15, such as comments regarding service, patient preferences, patient experiences, and the like. Any such information can be entered and associated with the patient and the patient device 15, and stored by the server 13 of the cloud-based computing infrastructure 12, such that it may be accessed in the future by facility personnel as a means of providing a more thoroughly personalized and informed patient experience.

The patient device 15 can take a number of different forms, including but not limited to a mobile device, smartphone, tablet computer, notebook computer, smart watch, or other computing device capable of supporting software applications and interacting with wireless networks, including the internet, and other wireless devices, including the location beacon(s) 16, as would be appreciated by those of skill in the art Likewise, the facility associated computing devices 17 can also take the form of a mobile device, smartphone, tablet computer, notebook computer, desktop computer, smart watch, or other computing device capable of operating a native mobile application or web-based application and interacting with wireless networks, including the internet, and other wireless devices, as would be well understood by those of skill in the art.

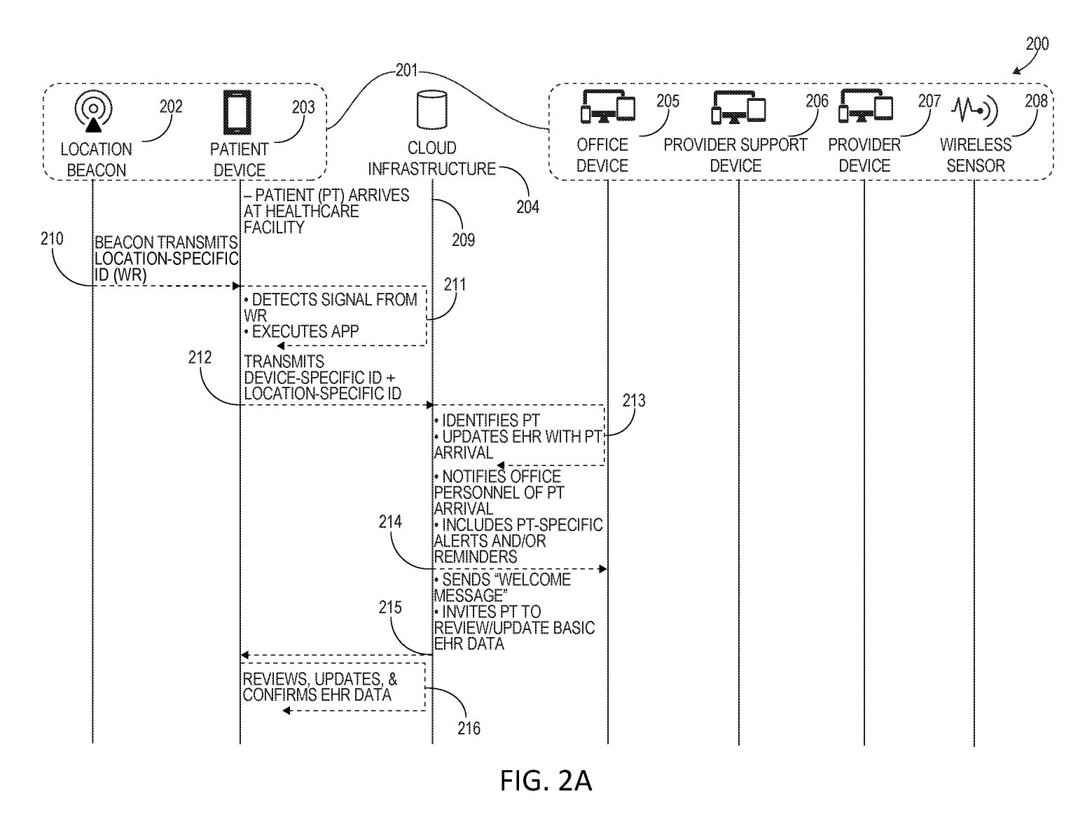

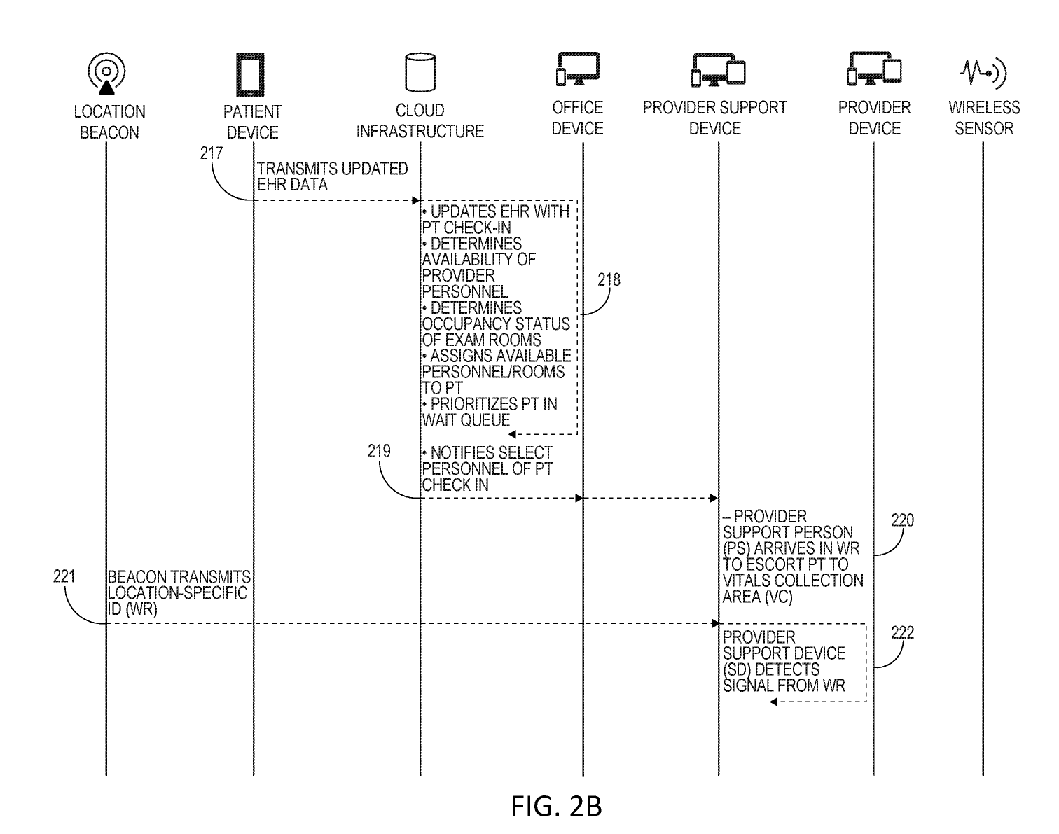

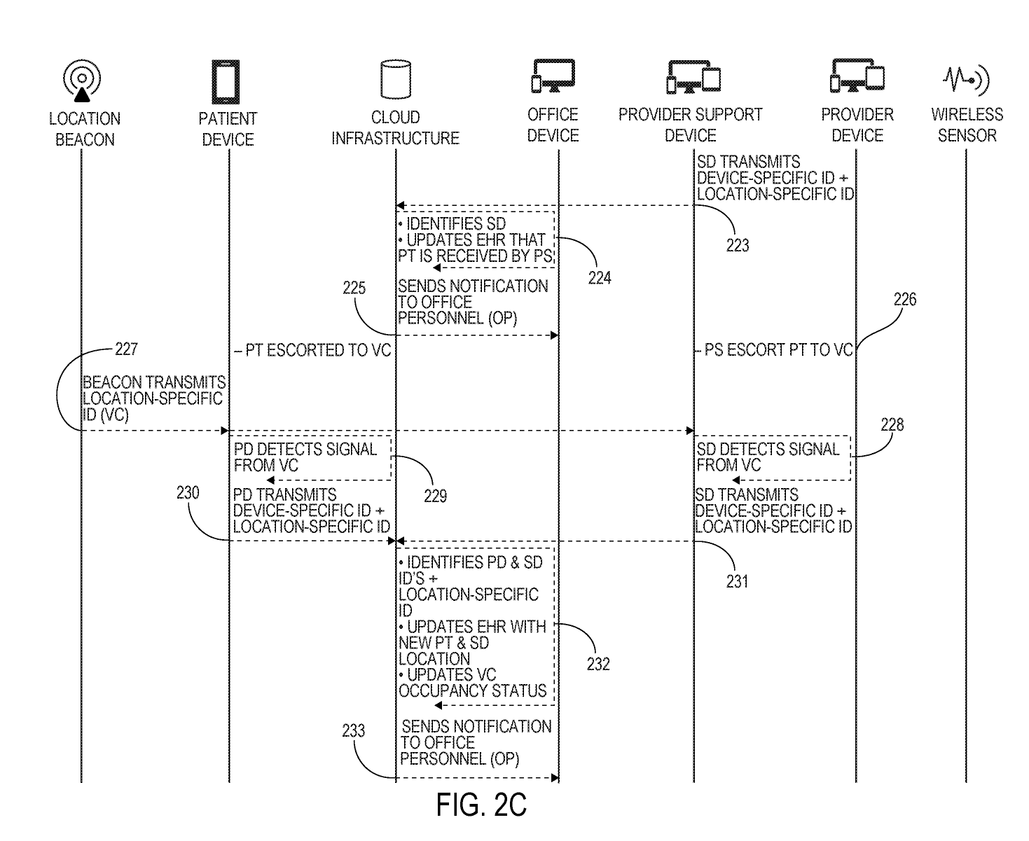

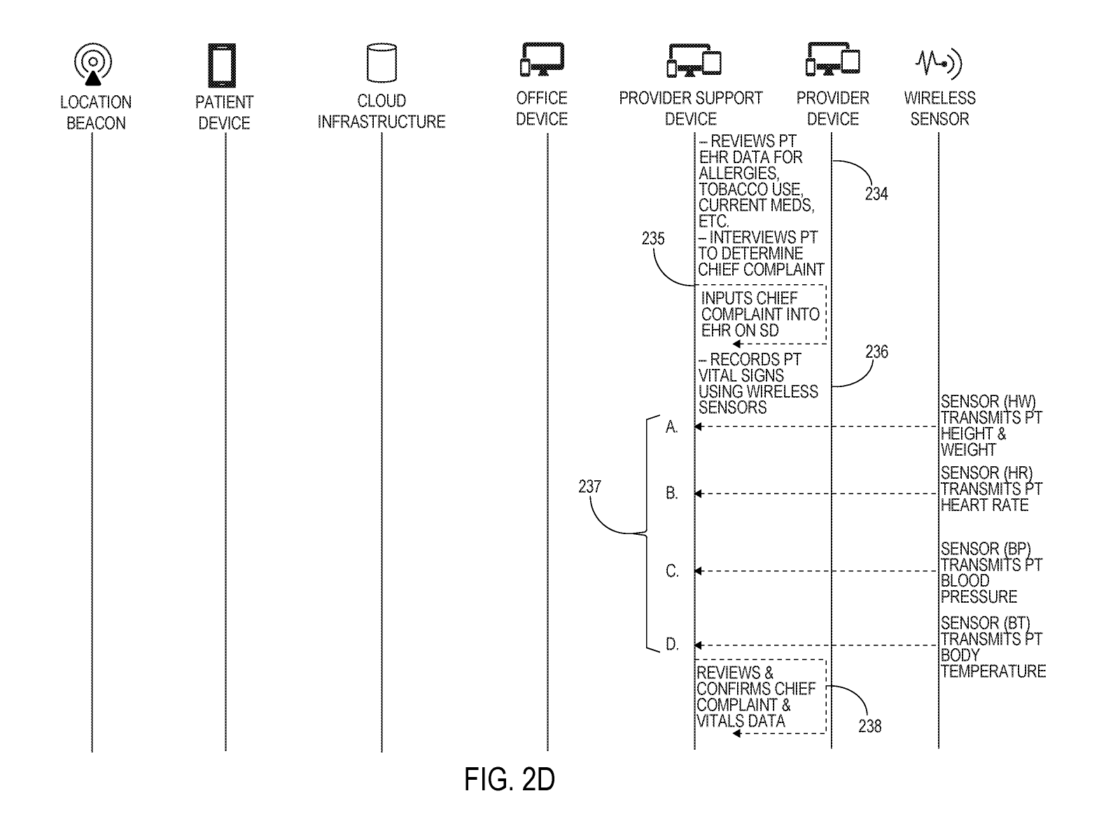

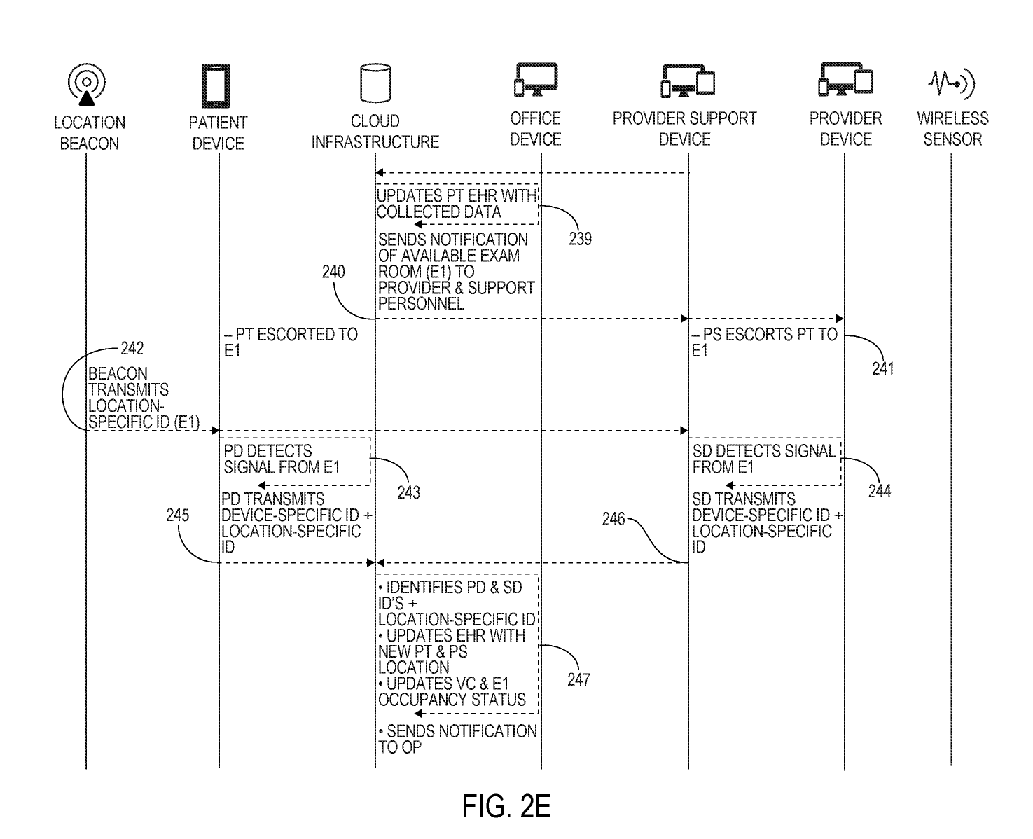

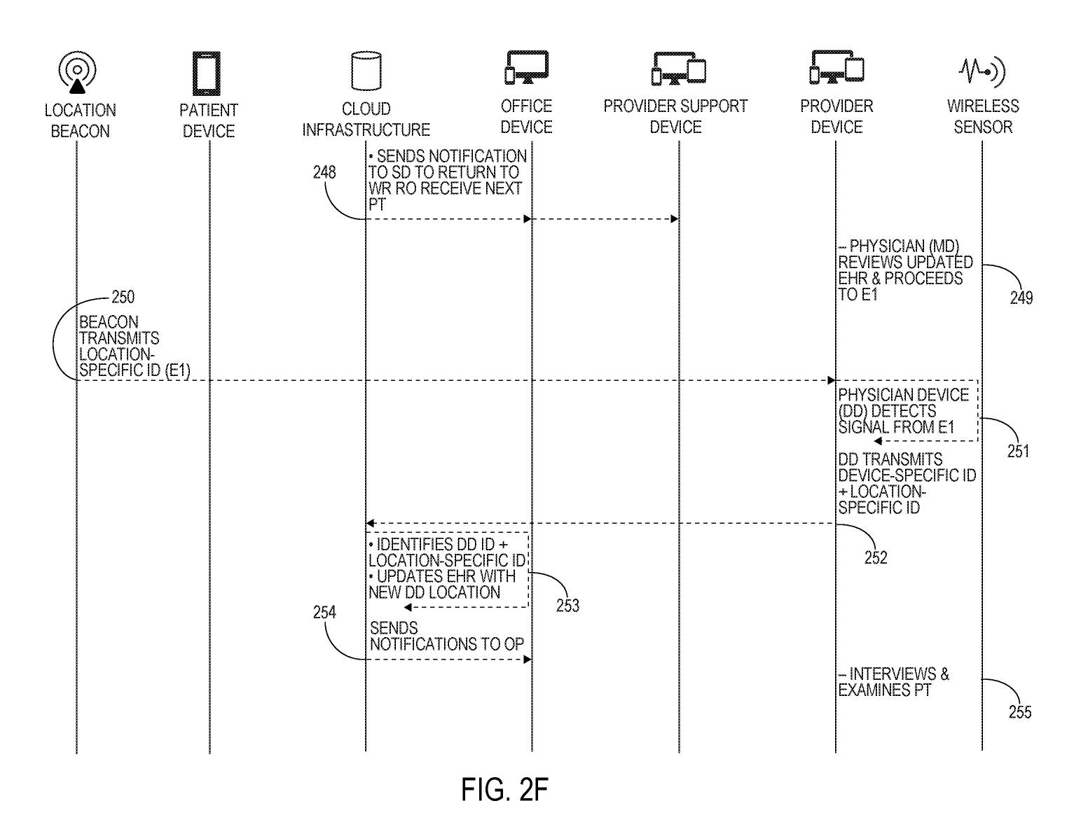

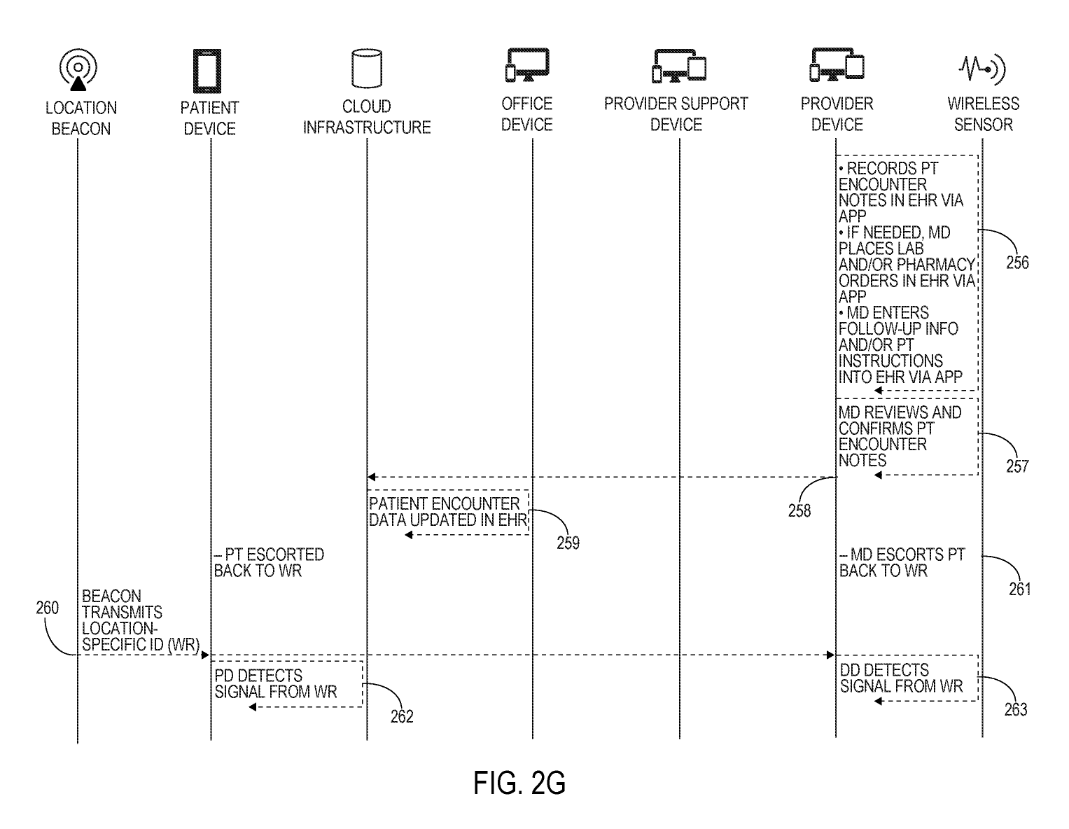

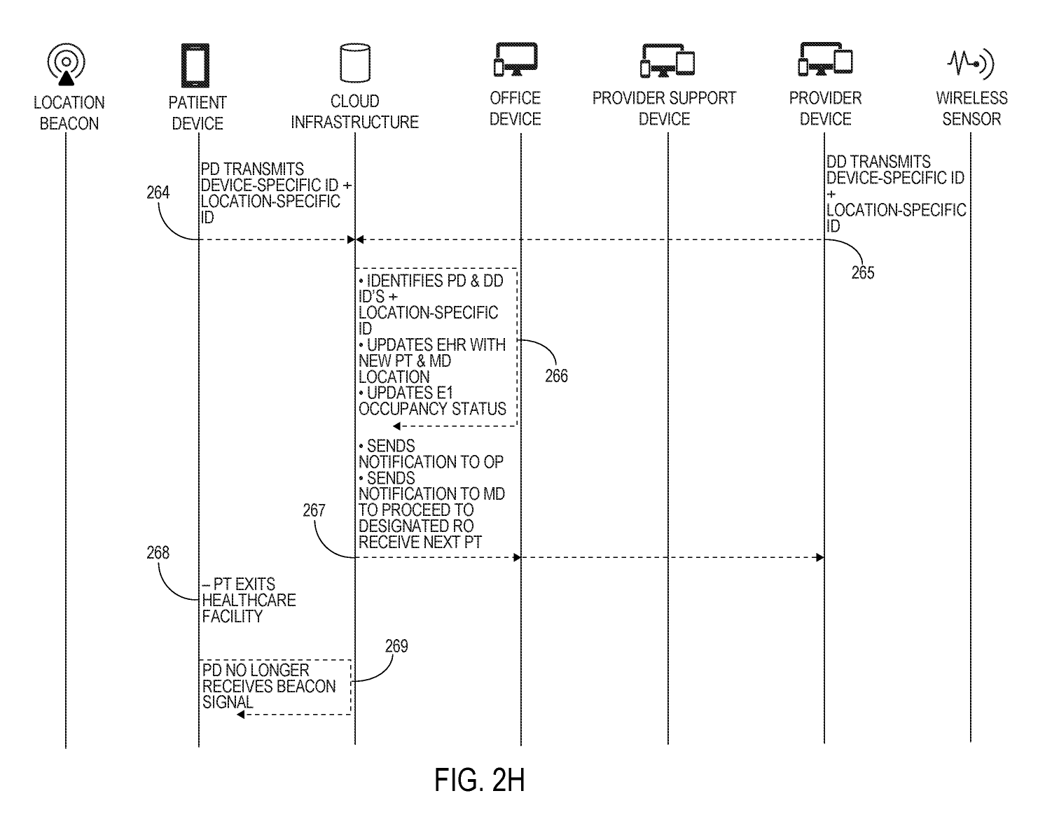

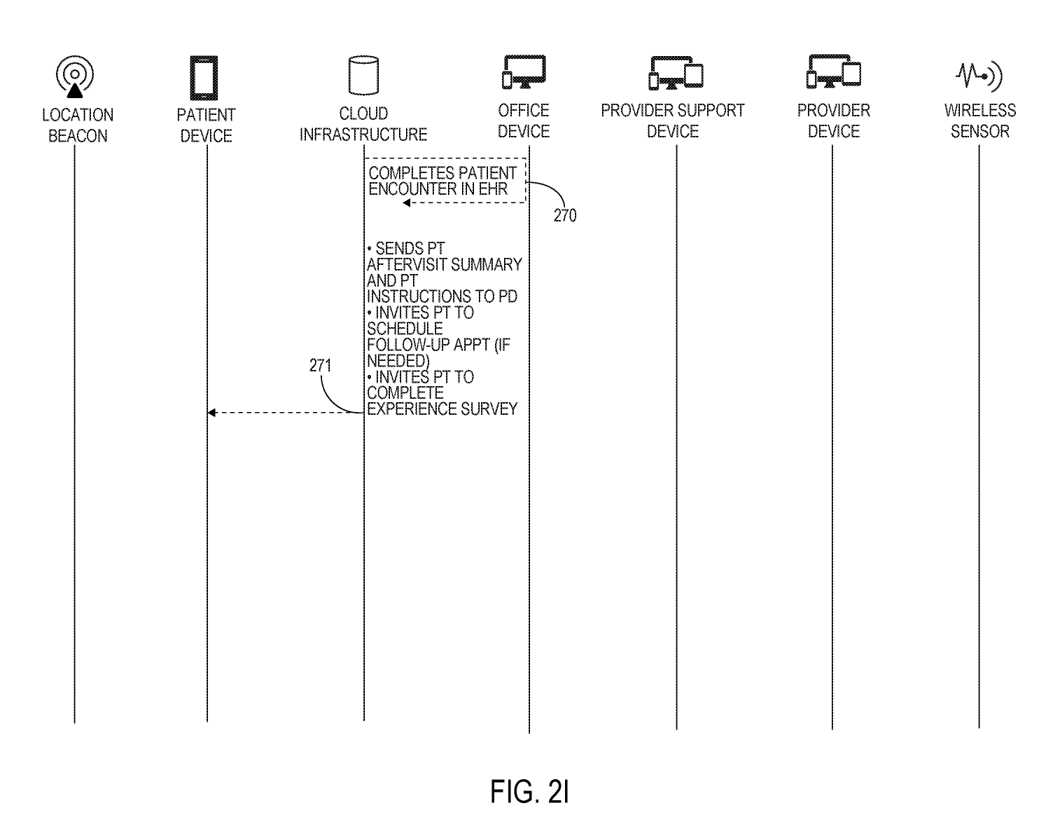

In operation, as shown in FIG. 2A, the patient with their patient device 203 enters the healthcare facility 201 (step 208). The patient device 203, located in the facility's waiting room, detects and communicates with the BLE-enabled location beacon 202 (step 210, 211). In accordance with an example embodiment, the location beacon 202 can broadcast data packets including a unique identifier and/or a website URL and the patient device 203 can detect the data packets (step 211) and save the unique identifier from the data packets. The data packets broadcast by the location beacon 202 convey the universally unique identifier specific to that facility (step 210), which itself communicates facility information, or alternatively enables the patient device 203 to obtain establishment information via, for example, the Internet. For example, the data packets provide the information necessary to obtain the unique identifier for the facility 201, as discussed with respect to FIG. 1. The application operating on the patient device 203 then executes (step 211) and transmits information about the patient (e.g., the device's unique IMEI or other information like a patient identifier assigned by the server) and the identification of the facility to the cloud-based computing infrastructure 204 (step 212). As would be appreciated by one skilled in the art, steps 210-214 can be performed automatically and transparently to the patient by the application on the patient device 203.

Continuing with FIG. 2A, the cloud-based computing infrastructure 204 transmits a notification to the facility's administrative office device(s) 205 (step 214), the notification including patient information and patient appointment information to include, if needed, patient-specific alerts and/or reminders. Simultaneously, the cloud-based computing infrastructure transmits a notification to the patient device 203, welcoming the patient to the facility and inviting the patient to review and/or update basic patient-specific EHR information, to possibly include recent changes in demographics, patient contact information, and insurance provider information (step 215). The patient can then make changes to the information stored in their EHR by following instructions and entering information into the application stored on the patient device 203 (step 216).

Referring to FIG. 2B, once the changes are made, the patient can then review, confirm, and submit the changes, that are transmitted to the cloud-based infrastructure for storage (step 217). With the patient's changes saved, the check-in process is complete. The server 13 can then poll facility-related devices to determine the availability of provider support staff, as well as the occupancy status of select areas/rooms within the facility equipped with location beacons 202 (step 218). Once availability of personnel is determined and area/room occupancy status verified, the server 13 can assign available personnel and areas/rooms to the patient (step 218). If desired, the server 13 can additionally prioritize the patient within the wait queue according to various factors such as appointment time, patient arrival time, check-in time, and urgency of care (step 218). Select personnel, e.g. office personnel and support staff, are then notified that the patient's check-in process is complete and that the patient's appointment is able to proceed (step 219). The patient's status information may include such information as position in the queue and estimated wait time, can be actively monitored by the patient using the application on the patient's device. In some embodiments, step 218 can be performed automatically by the cloud infrastructure 204 transparently to the patient.

If desired, as depicted in the present example, having received notification of the patient's arrival, the designated provider support staff (e.g., medical assistant, nurse, etc.) arrives in the waiting room to receive the assigned patient and escort them to the available vital signs collection area (step 220). As the support staff member arrives in the waiting room area, the support staff member's device 206 detects and communicates with the location beacon 202 (step 222) located there (i.e., the very same location beacon 202 detected by the patient device 203 in steps 210-211).

Referring to FIG. 2C, FIG. 2C illustrates a process that may be implemented by the system in FIG. 1. The support staff member's device 206 receives the unique identifying data from the location beacon 202 and relays this data in addition to the unique identification, i.e. IMEI, of the support staff member's device 206 to the cloud-based infrastructure 204 (step 223). The server 13 receives this identifying information and with it updates the EHR to show that the patient is now received in the waiting room by that particular support staff member (step 224). Steps 221-224 can be performed automatically by the application on the support staff member's device 206 transparently to the support staff member.

If desired, the server 13 can then inform select administrative support staff members (e.g., office personnel) that the patient is received by that particular support staff member by transmitting a notification to select administrative staff member's devices 205 (step 225). Then, having received the patient, the support staff member in this example may escort the patient to the facility's vital signs collection point, i.e., a designated room, hallway, or area where certain medical instruments are available which are designed to measure a patient's vital signs (step 226). As the patient and support staff member arrive at the vitals collection point, a different location beacon 202 there can wirelessly transmit a unique location-specific identification, to be detected by both the patient's device 203 and the support staff member's device 206 (steps 227-229). Each device 203, 206 then, having detected the location beacon's 202 unique identification, can relay the location-specific identification along with each device's 203, 206 unique identification to the cloud-based infrastructure 204 (steps 230-231), thus updating the server 13 with the patient's and support staff member's revised location. At the same time, the server 13 can determine that the vitals collection point is presently occupied and, if desired, send a notification of the updated location of the patient and support staff member, as well as the updated occupancy status of the vitals collection point to select administrative staff members, e.g. office personnel, devices 205 (steps 232-233).

Referring to FIG. 2D, From the application stored on the support staff member's device 206, the support staff member can then access the patient's EHR and review it for pertinent information such as patient allergies, tobacco use, and list of current medications (step 234). In this example, the support staff member then interviews the patient to determine the patient's chief complaint and reason for the appointment, e.g., abdominal pain, persistent cough, routine physical exam, etc. (step 234), and inputs the patient's chief complaint into the patient's EHR (step 235). The support staff member can then measure and record the patient's vital signs by employing a limited array of wireless digital medical instruments 208. Continuing with the example in FIG. 2, the support staff member can first measure the patient's height and weight using a digital physician's scale 208, which then wirelessly transmits the patient's height and weight data to the support staff member's device 206 (step 237A). Following this same example, the support staff member can then measure the patient's heart rate using a digital heart rate monitor 208, which then wirelessly transmits the patient's heart rate data to the staff member's device 206 (step 237B). Next, the support staff member can measure the patient's blood pressure using a digital blood pressure monitor 208, which then wirelessly transmits the patient's blood pressure data to the staff member's device 206 (237C). Additionally, or alternatively, the staff member can measure the patient's body temperature using a digital thermometer 208, which likewise wirelessly transmits the patient's body temperature data to the support staff member's device 206 (237D).

Referring to FIG. 2E, FIG. 2E illustrates the support staff member, having collected the necessary patient vital sign data on the support staff member's device 206, can view and review the patient's chief complaint and collected vitals data for completeness and accuracy within the application and submit the data to the cloud infrastructure 204 for storage in the patient's EHR (steps 238-239). Following this submittal, the server 13 can then determine that the vitals collection portion of the patient encounter is complete and sends a notification to the support staff member as well as the primary provider (e.g., physician, nurse practitioner, physician assistant, etc.) of the immediate availability of the designated exam room (step 240). The support staff member having received the above notification, can escort the patient to the designated exam room (step 241). As the patient and support staff member arrive at the designated exam room, a different location beacon 202 there can wireles sly transmit a unique location-specific identification, to be detected by both the patient's device 203 and the support staff member's device 206 (steps 242-244). Each device 203, 206 then, having detected the proximity sensor's 202 unique identification, can relay the location-specific identification along with each device's 203, 206 unique identification to the cloud-based infrastructure 204 (steps 245-246), thus updating the server 13 with the patient's and support staff member's revised location. Simultaneously, the server 13 can determine that the designated exam room is presently occupied and, if desired, send a notification of the updated location of the patient and support staff member, as well as the updated occupancy status of the designated exam room to select administrative support staff, i.e. office personnel, devices 205 (step 247-248). In some embodiments, the server 13 may determine and input into the cloud infrastructure 204 the result of the patient visit based on the exam rooms and the location of the patient. For example, when the patient device 203 is detected in the XRAY room or XRAY waiting room, the server 13 may determine that an XRAY was performed and the server 13 may trigger a radiology consultation appointment request for the staff members to approve or suggest at the time of the patient's departure.

Referring to FIG. 2F, FIG. 2F illustrates having received notification on the primary provider's device 207 of the patient waiting in the designated exam room, the primary provider can review the patient's updated EHR via an application stored on the primary provider's device 207 before proceeding to meet the patient (step 249). As the primary provider arrives at the designated exam room, the same location beacon 202 detected previously by the patient device 203 and staff member's device 206, can wirelessly transmit a unique location-specific identification, to be detected by the primary provider's device 207 (steps 250-251). The provider device 207 then, having detected the location beacon's 202 unique identification, can relay the location-specific identification along with the device's 207 unique identification to the cloud-based infrastructure 204 (step 252), thus updating the server 13 with the primary provider's revised location. Simultaneously, the server 13 can determine that the designated exam room is presently occupied and, if desired, send a notification of the updated location of the patient and primary provider, as well as the updated occupancy status of the designated exam room to select administrative personnel, e.g. office personnel, devices 205 (step 253-254).

Having already reviewed the patient's updated EHR (step 249), in this example, the primary provider can then interview and examine the patient as needed to achieve a proper diagnosis as it relates to the patient's chief complaint (step 255).

Referring to FIG. 2G, FIG. 2G illustrates the patient recording the visit notes into the application stored on the primary provider's device 207 (step 256). The primary provider can then, if needed, place laboratory and/or pharmacy orders for the patient via the application stored on the primary provider's device 207. Similarly, when the visit is determined completed, the primary provider can enter appointment follow-up information and/or special patient instructions into the application stored on the primary provider's device 207 (step 256).

The primary provider can review the patient visit notes, orders, summary, and instructions for accuracy and completeness before submitting it from the primary provider's device 207 to the cloud infrastructure 204 to be stored in the patient's EHR (step 258-259). Once submitted, the primary provider can then escort the patient back to the waiting room, where the location beacon 202 there can wirelessly transmit a unique location-specific identification, to be detected by both the patient's device 203 and the primary provider's device 207 (steps 260-263).

Referring to FIG. 2H, FIG. 2H illustrates each device 203, 207, having detected the location beacon's 202 unique identification, can relay the location-specific identification along with each device's 203, 207 unique identification to the cloud-based infrastructure 204 (steps 264-265), thus updating the server 13 with the patient's and primary provider's revised location. At the same time, the server 13 can determine that the exam room is presently unoccupied and, if desired, send a notification of the updated location of the patient and primary provider, as well as the updated occupancy status of the exam room to select administrative support, e.g. office personnel, devices 205 (steps 266-267). Simultaneously, if desired, the server 13 can send a notification to the primary provider's device 207, notifying the primary provider to proceed to the designated location of the next patient (step 267).

As the patient exits the healthcare facility 201 with the patient device 203 and travels outside the range of the location beacon 202 located in the waiting room (step 269).

Referring to FIG. 21, FIG. 21 illustrates the server 13 that is configured to determine that the patient encounter is complete and conclude the appointment in the patient's EHR (step 270). The appointment having been concluded, the server 13 can then send to the patient device 203 an after-visit summary, including any patient instructions previously input by the primary provider, as well as an invitation and instructions on how to schedule a follow-up appointment (if needed), and an invitation and instructions on how to complete a patient experience survey (step 271).

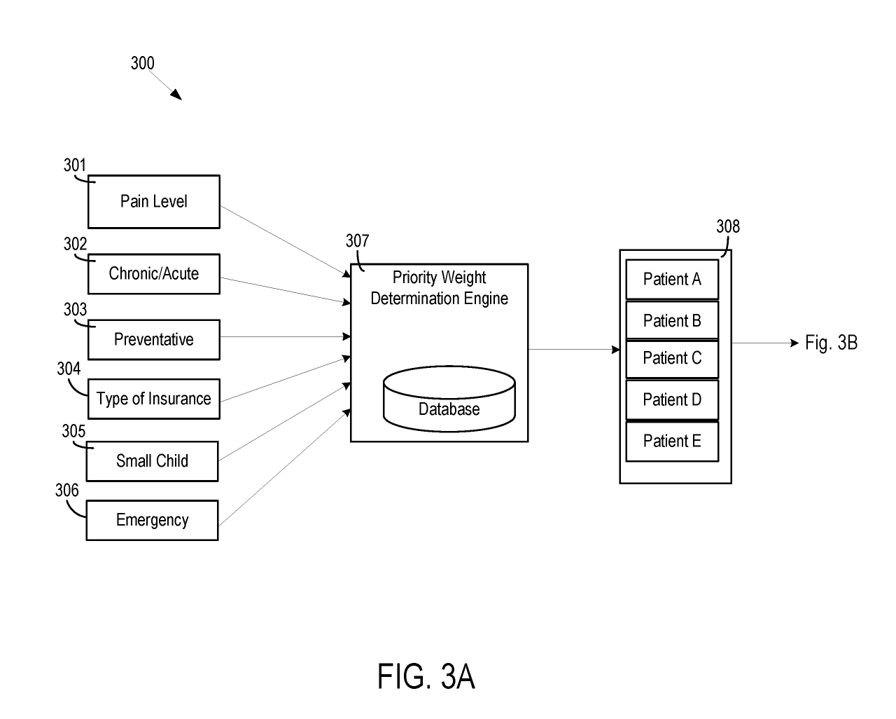

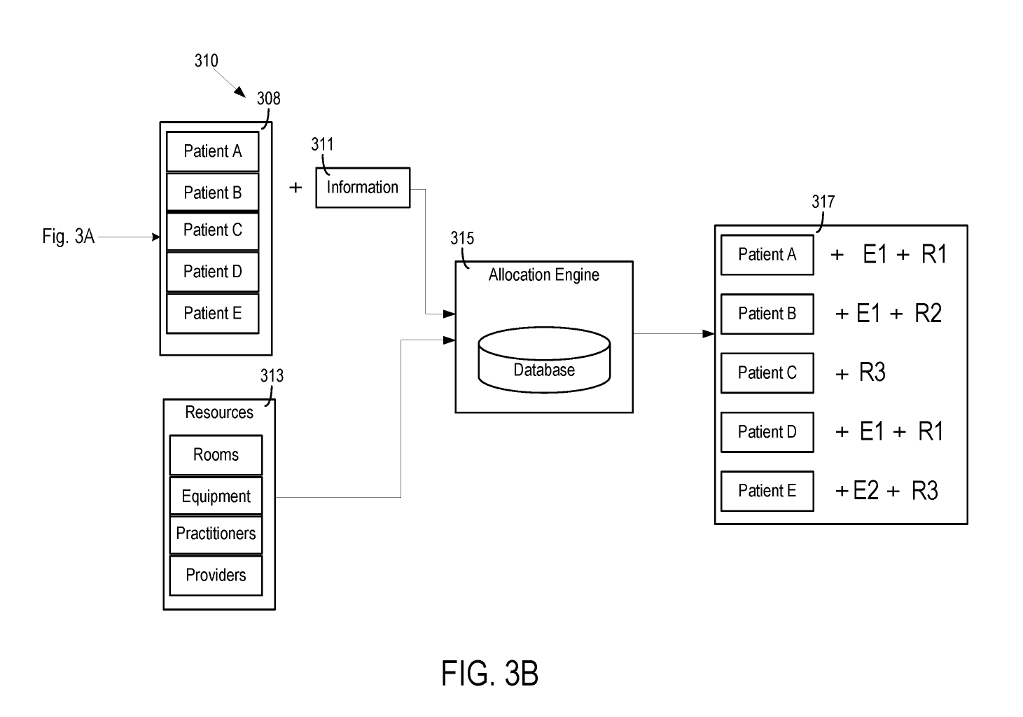

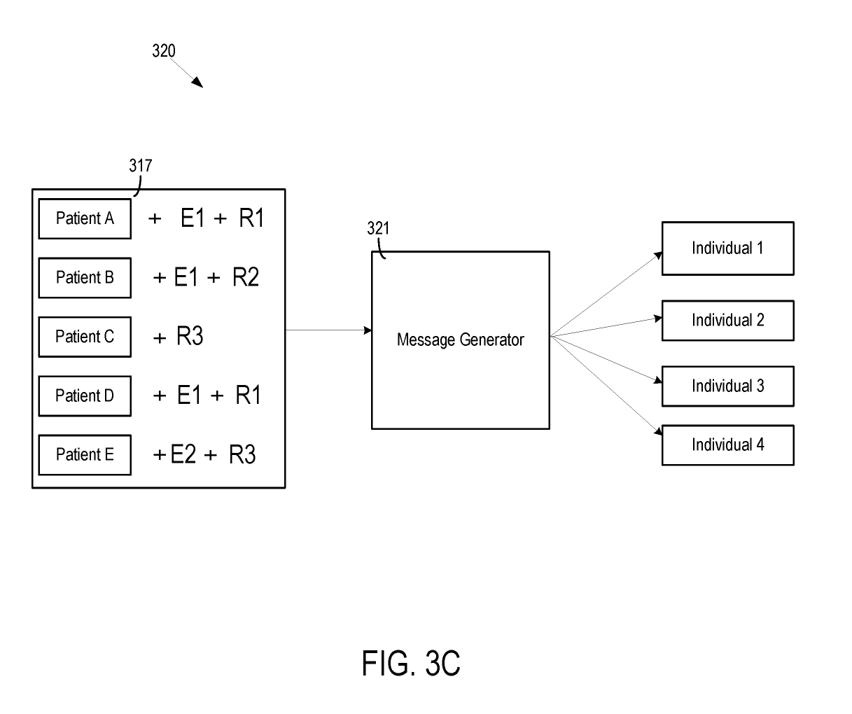

Referring to FIG. 3A, FIG. 3A illustrates a process 300 that may be implemented by the system in FIG. 1. When determining the patient prioritization a priority weight determination engine 307 may be configured to receive various parameters for input such as, but not limited to, pain level 301, chronic/acute 302, preventative care 303, type of insurance 304, small child 305, and emergency level 306. Based on one or more of the above parameters the priority weight determination engine 307 is configured to prioritize the patients that are waiting or about to enter the health care facility. In various embodiments, the priority weight determination engine 307 may rank the parameters (pain level 301, chronic/acute 302, preventative 303, types of insurance 304, small child 305, and emergency 306). For example, the pain level 301 may be ranked the highest or 6 out of the 6 listed parameters or emergency may be ranked the highest. In some embodiments, the health care facility personnel may determine the ranking for each parameter. For example, in a pediatrician's office small child 305 may not be ranked since all of the patients are small children. Accordingly, the system from FIG. 1 may determine the characteristics of all of the patients and rank the parameters based on the characteristics. In other embodiments, the priority weight determination engine 307 may rate chronic/acute 302 as the lowest rank 1. Each patient's parameter ranking may be added up to determine a score for each patient that determines their placement in the patient prioritization table 308.

Continuing with FIG. 3A, the priority weight determination engine 307 may determine a patient prioritization table 308 (Patients A, B, C, D, and E). The patient prioritization table 308 may be determined based on parameter rankings.

Referring to FIG. 3B, FIG. 3B illustrates a process 310 that may be implemented by the system in FIG. 1. The patient prioritization table 308 may be combined with information 311 that may is provided by the patients. The information 311 may include the patient provided information that may be provided to the allocation engine 315. The patient prioritization table 308, information 311 and resources 313 may be provided to the allocation engine 315. The resources 313 may be resources that are available to the healthcare facility that may include, for example, rooms, equipment, practitioners, providers, personnel, and nurses.

The allocation engine 315 may receive and process resources 313, information 311 and allocation table 308 to determine an allocation order 317. As shown in FIG. 3B, the allocation order 317 may include patient name and resources. The allocation engine 315 may allocate resources 313 based on the patient prioritization table 308. For example, patient A may be assigned room 1 with equipment 2, patient B may be assigned to room 2 with equipment 1, patient C may be assigned room 3 and only the equipment that is in room 3, patient D may be assigned room 1 after patient A has finished using room 1.

Referring to FIG. 3C, FIG. 3C illustrates a process 320 that may be implemented by the system in FIG. 1. In particular, the message generator 321 may receive the allocation table 319 and generate messages to personnel regarding actions to be taken. For example, a message may be generated to the office personnel 1 to take patient A to exam room 1 and take vital information. Other messages may be sent to office personnel 2 to take equipment 1 to room 2 and use it on patient 1.

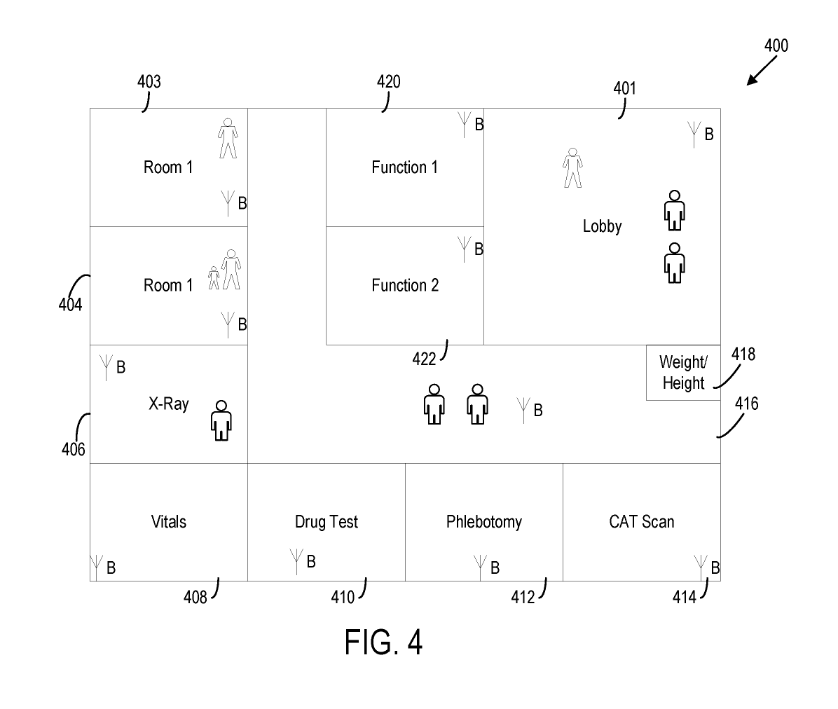

Referring to FIG. 4, FIG. 4 is a schematic of an office diagram 400 representing an example healthcare facility with a system from FIG. 1. The office from the diagram 400 may include lobby 401, rooms 403, 404, 406, 408, restroom 410, phlebotomy room 412, CAT scan room 414, hallway 416, weighing scale 418, function room 420 and function room 422. Each of the above-mentioned rooms and areas can have a location beacon B. The beacons shown in FIG. 4 may track each of the individuals shown in FIG. 4. FIG. 4 may also provide various other analytics services discussed below.

Various embodiments include determining the physical location of multiple patients by the patients' mobile devices 203 within a healthcare facility relative to particular location beacons, thereby determining the distribution of patients among the various locations within the facility, e.g. exam rooms, waiting room, doctors' offices, etc. Other embodiments include the ability to determine the physical location of various care provider personnel by the care providers' mobile devices within a healthcare facility relative to particular location beacons, thereby determining the distribution of provider personnel among the various locations within the facility, e.g. exam rooms, waiting room, doctors' offices, etc.

Various embodiments include determining by way of digital time stamps, location beacon signals, and spatial-temporal data received from patient and provider mobile devices, the movement patterns of patients and provider personnel as they maneuver among and between various locations within a facility. The system disclosed in FIG. 1 may be configured to provide analytics reporting of collected data and dashboard representation of real-time data received from patient and provider mobile devices, allowing for high level as well as detailed analysis of operational workflows.

Various embodiments allow patients the option of “checking in” to their appointment electronically by using a native application stored on their mobile device, thus eliminating the need to interact with and share private health-related information with front office personnel.

In other embodiments, the system is configured to automatically determine the conclusion of a patient appointment using data received from a patient's mobile device in communication with location-specific beacon signals.

In other embodiments, the system is configured to streamline and automate the process of collecting patient vitals data by recording digitally measured vitals data wireles sly directly into a patient's electronic health record from specially-equipped wireless medical instruments. The system in FIG. 1 may be configured to notify administrative and provider personnel of a facility of the arrival of a patient by way of the patient's mobile device in communication with specific location beacons, then organize and sort patients in a wait queue according to various factors such as arrival time, appointment time, and urgency of care.

Various embodiments, allow provider personnel to submit appointment summary information, patient care instructions, follow-up appointment scheduling details, and patient satisfaction survey requests, electronically to patients directly upon completion of their appointment by way of electronic notification using means such as email, text message, patient portal, etc. The system shown in FIG. 1 may collect data related to patient appointment arrival times, cancelations, walk-ins, “no-shows,” and discrepancies between forecasted and actual appointment durations, such that providers and administrative personnel can analyze and maximize scheduling and resource allocation efficiency.

What has been described above includes examples of the systems and methods. It is, of course, not possible to describe every conceivable combination of components or methodologies for purposes of describing the subject innovation, but one of ordinary skill in the art may recognize that many further combinations and permutations of the innovation are possible. Accordingly, the innovation is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

The embodiments of the present disclousre have been described with reference to drawings. The drawings illustrate certain details of specific embodiments that implement the systems and methods and programs of the present invention. However, describing the invention with drawings should not be construed as imposing on the invention any limitations that may be present in the drawings. The present invention contemplates methods, systems and program products on any machine-readable media for accomplishing its operations. The embodiments of the present invention may be implemented using an existing computer processor, or by a special purpose computer processor incorporated for this or another purpose or by a hardwired system.

As noted above, embodiments within the scope of the present invention include program products comprising non-transitory machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. Combinations of the above are also included within the scope of machine-readable media. Machine-executable instructions comprise, for example, instructions and data which cause a general purpose computer, special purpose computer, or special purpose processing machines to perform a certain function or group of functions.

Embodiments of the present invention have been described in the general context of method steps which may be implemented in one embodiment by a program product including machine-executable instructions, such as program code, for example in the form of program modules executed by machines in networked environments. Generally, program modules include routines, programs, objects, components, data structures, etc. that perform particular tasks or implement particular abstract data types. Machine-executable instructions, associated data structures, and program modules represent examples of program code for executing steps of the methods disclosed herein. The particular sequence of such executable instructions or associated data structures represent examples of corresponding acts for implementing the functions described in such steps.

As previously indicated, embodiments of the present invention may be practiced in a networked environment using logical connections to one or more remote computers having processors. Those skilled in the art will appreciate that such network computing environments may encompass many types of computers, including personal computers, hand-held devices, multi-processor systems, microprocessor-based or programmable consumer electronics, network PCs, minicomputers, mainframe computers, and so on. Embodiments of the invention may also be practiced in distributed computing environments where tasks are performed by local and remote processing devices that are linked (either by hardwired links, wireless links, or by a combination of hardwired or wireless links) through a communications network. In a distributed computing environment, program modules may be located in both local and remote memory storage devices.

An exemplary system for implementing the overall system or portions of the invention might include a general purpose computing computers in the form of computers, including a processing unit, a system memory, and a system bus that couples various system components including the system memory to the processing unit. The system memory may include read only memory (ROM) and random access memory (RAM). The computer may also include a magnetic hard disk drive for reading from and writing to a magnetic hard disk, a magnetic disk drive for reading from or writing to a removable magnetic disk, and an optical disk drive for reading from or writing to a removable optical disk such as a CD ROM or other optical media. The drives and their associated machine-readable media provide nonvolatile storage of machine-executable instructions, data structures, program modules and other data for the computer. It should also be noted that the word “terminal” as used herein is intended to encompass computer input and output devices. Input devices, as described herein, include a keyboard, a keypad, a mouse, joystick or other input devices performing a similar function. The output devices, as described herein, include a computer monitor, printer, facsimile machine, or other output devices performing a similar function.

It should be noted that although the diagrams herein may show a specific order and composition of method steps, it is understood that the order of these steps may differ from what is depicted. For example, two or more steps may be performed concurrently or with partial concurrence. Also, some method steps that are performed as discrete steps may be combined, steps being performed as a combined step may be separated into discrete steps, the sequence of certain processes may be reversed or otherwise varied, and the nature or number of discrete processes may be altered or varied. The order or sequence of any element or apparatus may be varied or substituted according to alternative embodiments. Accordingly, all such modifications are intended to be included within the scope of the present invention as defined in the appended claims. Such variations will depend on the software and hardware systems chosen and on designer choice. It is understood that all such variations are within the scope of the invention. Likewise, software and web implementations of the present invention could be accomplished with standard programming techniques with rule based logic and other logic to accomplish the various database searching steps, correlation steps, comparison steps and decision steps.

The foregoing description of embodiments of the invention has been presented for purposes of illustration and description. It is not intended to be exhaustive or to limit the invention to the precise form disclosed, and modifications and variations are possible in light of the above teachings or may be acquired from practice of the invention. The embodiments were chosen and described in order to explain the principals of the invention and its practical application to enable one skilled in the art to utilize the invention in various embodiments and with various modifications as are suited to the particular use contemplated. Other substitutions, modifications, changes and omissions may be made in the design, operating conditions and arrangement of the embodiments without departing from the scope of the present invention as expressed in the appended claims.

1. A computer system, comprising:
one or more processors; and
non-transitory machine readable storage media coupled to the one or more processors and having instructions stored therein that are executable by the one or more processors, including an electronic module configured to:
receive, by a location beacon, a signal from a mobile device that includes a device specific identifier and location specific identifier;
determine an identity of an owner of the mobile device;
update electronic health record with a location specific identifier of the mobile device;
generate a notification message to the mobile device and an alert for other individuals based on information regarding the owner of the mobile device.

2. The computer system from claim 1, wherein the electronic module is configured to send the notification message to a device accessible to other individuals.

3. The computer system from claim 1, wherein the electronic module is configured to determine the movement pattern of the mobile device by digital time stamp, location beacon, spatial-temporal data received from the mobile device.

4. The computer system from claim 1, wherein the electronic module is configured to provide analytics reporting to collected data and dashboard representation of real-time data received from the owner of the mobile device

5. The computer system from claim 1, wherein the electronic module is configured to collect data related to arrival times, cancelations, walk-ins based on the mobile device.

6. The computer system from claim 1, wherein the electronic module is configured to determine a physical location of multiple patients by using their mobile devices.

7. A method for updating a medical record, comprising:
determining, using a first location mechanism, a patient location within a facility;
determining, using a second location mechanism, care provider location relative to the patient location;
responsive to determining the patient location relative to the patient location, determining the conclusion of a patient appointment using data received from a mobile device and a location specific beacon within the facility.

8. The method of claim 7, wherein the data received from the mobile device and the location specific beacon includes temporal data that calculates a period of time that the patient location and the care provider location are within a proximity of each other.

9. The method of claim 8, wherein the period of time calculation begins when the patient location and the care provider location is within about 6 feet to 10 feet of each other.

10. The method of claim 8, wherein the period of time calculation begins upon determining that a patient device is in NFC communication with a care provider device.

11. The method of claim 8, wherein the period of time calculation begins upon determining that a patient device is in communication with the location specific beacon and a care provider device is in communication with the location specific beacon.

12. The method of claim 8, wherein the conclusion of a patient appointment is determined based on information received by the location specific beacon during a period of time data received from a mobile device and a location specific beacon within the facility.

13. The method of claim 7, further comprising determining a movement pattern of the patient location using a digital time stamp, location specific beacon, and spatial-temporal data.

14. The method of claim 7, further comprising notifying administrative and provider personnel of a facility of the arrival of the patient response to receiving communication form a patient mobile device.

15. A computer system, comprising:
one or more processors; and
non-transitory machine readable storage media coupled to the one or more processors and having instructions stored therein that are executable by the one or more processors, including an electronic module configured to:
receive, by a location beacon, a signal from one or more mobile devices belonging to a first set of individuals that each include a device specific identifier and location specific identifier;
determining the physical location of the one or more mobile devices within a facility relative to the location beacon, thereby determining the distribution of mobile device among the various locations within the facility.

16. The computer system of claim 15, further comprising the electronic module configured to determine the physical location of one or more mobile devices belonging to a second group of individuals within a facility relative to the location beacons, thereby determining the distribution of the second group of individuals.

17. The computer system of claim 15, further comprising the electronic module configured to determine by way of digital time stamps, location beacon signals, and spatial-temporal data received from the mobile devices, the movement patterns of the first set of individuals and a second set of individuals as they maneuver among and between various locations within the facility.

18. The computer system of claim 15, further comprising the electronic module configured to generate analytics reporting of collected data received from the first set of individuals and the second set of individuals.

19. The computer system of claim 15, further comprising the electronic module configured to allow the first set of individuals to checking in for their appointment electronically by using a native application stored on their mobile device by eliminating the need to interact with and share private health-related information with the second set of individuals

20. The computer system of claim 15, wherein the electronic module is configured to collect data related to arrival times, cancelations, walk-ins based on the one or more mobile devices.

21. A method for establishing electronic communication comprising:
receiving from a between a patient device information regarding the patient location and a patient identified payer;
determining the patient device is located at a point of care facility where the patient may be examined by a medical provider using an Electronic Health Record System;
triggering an electronic communication ability between payer and patient based on the real time assessment by the medical provider.

22. The method of claim 21, wherein the payer is a health insurance company. Enabling health insurance company or its affiliate to communicate directly with the patient to help manage patient disease.
